(19) United States
(12) Patent Application Publication
(10) Pub. No.: US 2012/0150218 A1
(43) Pub. Date: Jun. 14, 2012

(54) MEDICAL DEVICE FOR OCCLUDING A HEART DEFECT AND A METHOD OF MANUFACTURING THE SAME

(76) Inventors: Robert Tyler Sandgren, Lindstrom, MN (US); Gary Erzberger, Minneapolis, MN (US); Dara Chin, St. Paul, MN (US); Michael Patrick Corcoran, Woodbury, MN (US)

(21) Appl. No.: 13/071,868

(22) Filed: Mar. 25, 2011

Related U.S. Application Data
(63) Continuation-in-part of application No. 12/387,918, filed on May 8, 2009.
Application No. 12/387,918 is a continuation-in-part of application No. 11/900,838, filed on Sep. 13, 2007.

(51) Int. Cl. A61B 17/08; B21F 45/00; B23P 11/00

(52) U.S. Cl. 606/213; 29/428

(57) ABSTRACT

An implantable device for occluding a septal defect has interleaved frame sections that allow flexibility to conform to a variety of defect geometries and provide reliable occlusion during endothelialization. Left and right frames connect to opposite ends of a floating connection post. The device is resiliently deformable and is biased into a natural state wherein, in situ in a variety of defect geometries, the device applies a sandwiching force to the tissue surrounding the defect that is relatively uniform across its diameter, improving stability and promoting occlusion.

This application is a continuation-in-part of U.S. Ser. No. 12/387,918, filed May 8, 2009, entitled Medical Device for Occluding a Heart Defect and a Method of Manufacturing the Same, and is a continuation-in-part of U.S. Ser. No. 11/900,838, filed Sep. 13, 2007, entitled Occlusion Device with Centering Arm Network, both of which are incorporated herein in their entirety.

### FIELD OF THE INVENTION

The present invention relates generally to an occlusion device for closing an aperture in a biological structure and more particularly for closing a conduit or aperture in a heart wall, such as a defect between atrial chambers.

### BACKGROUND OF THE INVENTION

The heart is comprised, generally, of four chambers: the left and right atria and the left and right ventricles. Separating the left and right sides of the heart are two walls or “septa”. The septa are susceptible to a number of types of defects, including patent ductus arteriosus, patent foramen ovale, atrial septal defects and ventricular septal defects. Although the causes and physical characteristics of these defects vary by type, they generally involve an opening (e.g. an aperture, slit, conduit, flap-covered aperture) through the septum that allows blood to shunt between chambers in the heart in an abnormal way that compromises the performance of the heart and circulatory system and has disadvantageous health consequences.

The defect in the septum can be surgically repaired via open heart surgery that requires a patient to undergo general anesthesia and requires opening of the chest cavity. Open-heart surgery is relatively risky, painful and expensive. An open-heart patient may spend several days in a hospital, will experience considerable pain, will take several weeks to recover before being able to return to normal activities, and will carry a large, prominent scar.

To avoid the risks and discomfort associated with open heart surgery, modern occlusion devices have been developed that are small, implantable devices capable of being delivered to the heart through a catheter. The delivery catheter is deployed through a relatively small incision through which it enters a major blood vessel. The catheter is snaked through the blood vessel to the heart where the occlusion device is deployed via remote (i.e. outside the body) manipulations by the doctor or cardiologist. This procedure is performed in a cardiac catheterization lab and avoids the risks, pain and long recovery associated with open heart surgery.

### SUMMARY OF THE INVENTION

There has been a need to improve occlusion devices to provide an easily deployable device that adapts well to a wide range of geometries, sizes, and types of defects. There has been a need for an occlusion device that centers itself within the defect, provides a reliable seal and maintains its position blocking the defect over days or weeks while the device is endothelialized (or covered by the growth of tissue). What has further been needed is an occlusion device that holds its position within the defect reliably without unduly squeezing or pinching adjacent tissue, since such squeezing can damage the tissue.

It has further been a need for the occlusion device to be retrievable so that if it is not placed initially as desired during its implantation procedure, the doctor can remove it via the catheter without damaging the device and without undue time and effort. Still further, there has been a need for an occlusion device that is easily loaded into a catheter, is easily deployed and is easily retracted back into the catheter and redeployed without removing it from the catheter for reloading so that the redeployment can be accomplished with the catheter in situ.

An occlusion device is described herein that meets these needs. The occlusion device of the present invention has left and right frames that each support a sheet. In broad terms, these left and right frames form flanges that, in situ, overlap tissue adjacent the defect and sandwich this tissue between them. A portion of the device extends through the defect.

The left frame is formed of splines that form a series of petals. These petals aid in distributing forces relatively uniformly about the periphery of the left frame.

In one embodiment, the right frame has a set of centering limbs and a set of arms. Each limb is linked to a corresponding arm. The right sheet is coupled to the arms. In a second embodiment, the right frame is formed of splines that form a series of petals. These splines further form internal limbs. The petals and limbs are formed by a series of looped wires, with each wire forming a portion of a limb and a portion of a petal.

The left frame is coupled to a connecting post. The centering limbs of the right frame are also coupled to the connecting post. More specifically, the connecting post has left and right ends; the splines of the left frame are coupled to the right end of the connecting post and the limbs of the right frame are coupled to the left end of the connecting post, such that the left and right frames are interleaved or cross over one another. This arrangement yields a particularly advantageously deformable construction that allows the device to adapt to defects of a variety of sizes, shapes and configurations.

The device is resiliently deformable through a range of positions from a collapsed, delivery shape that fits within a delivery catheter to an expanded, deployed configuration, with the frame-supported sheets radiating generally outward to form flanges to sandwich tissue therebetween. The device is biased into the deployed configuration. The distance between the frame-supported sheets is variable and is determined, in situ, by the thickness of the walls of the heart adjacent the defect. The device is spring-biased toward a configuration with the frame-supported sheets immediately adjacent one another, and this bias exerts sandwiching force on the adjacent tissue. However, the device can be elongated in response to applied force to increase the distance between the sheets to accommodate varying wall thicknesses. Further, the resiliency of the frames and the manner in which they attach to the connecting post allows the frame-supported sheets to tilt with respect to one another and/or to be axially offset from one another while still reliably and effectively occluding the defect.

### BRIEF DESCRIPTION OF THE DRAWINGS

An exemplary version of an occlusion device is shown in the figures wherein like reference numerals refer to equivalent structure throughout, and wherein:

FIG. 1 is perspective view of an exemplary embodiment of an occlusion device according to the present invention;

FIG. 2 is an end view of the device of FIG. 1 taken from the right side;

FIG. 3 is an end view of the device of FIG. 1, taken from the left side, i.e. from the opposite direction of the view of FIG. 2;

FIG. 4 is a perspective view of the device of FIG. 1, under axial force;

FIG. 5 is an enlarged, partial view of the device of FIG. 1;

FIG. 6 is an enlarged schematic view of the device of FIG. 1 in situ within a heart defect;

FIGS. 7a and 7b are schematic views of the device of FIG. 1 in situ within heart defects of different wall thicknesses and showing the distribution of forces applied by the device to tissue adjacent the defect;

FIG. 8a depicts the force distribution of prior art devices on tissue adjacent a heart defect;

FIGS. 9a-9c are schematic representations of limbs of the device of FIG. 1 adapting to defects of varying cross-sectional shapes;

FIGS. 10a-c are schematic representations of the device of FIG. 1 adapting to defects of varying geometries;

FIGS. 11a-f show the device of FIG. 1 being deployed via a catheter;

FIGS. 12a and 12b show alternate embodiments of links the connect limbs to radial arms in the device of FIG. 1; and

FIG. 13 is a schematic view of the device;

FIG. 14 is an enlarged perspective view of a PFO version of the device;

FIG. 15 is an enlarged perspective exploded view of the device illustrating various elements of the design;

FIG. 16 is a perspective view of the device;

FIG. 17 is an end view of the device shown without sheets and with the left frame in the foreground;

FIG. 18 is an end view of the right frame portion of the device;

FIG. 19 is an end view of the device with the right frame in the foreground;

FIG. 20 is view of a portion of the right frame the device shown without a sheet;

FIG. 21 is a view of the same portion of the right frame of the device as is depicted in FIG. 20, but with a sheet attached;

FIG. 22 is an end view of the device with the left side in the foreground;

FIG. 23a-f show the routing of elements of the device during assembly;

FIG. 24 is a view of a cross-section, taken along line 24-24 in FIG. 23a, of one post showing orientation of holes therethrough; and,

FIG. 25 is a view of a cross-section, taken along line 25-25 in FIG. 23a, of a second post showing the orientation of holes therethrough.

### DETAILED DESCRIPTION OF PREFERRED EMBODIMENT(S)

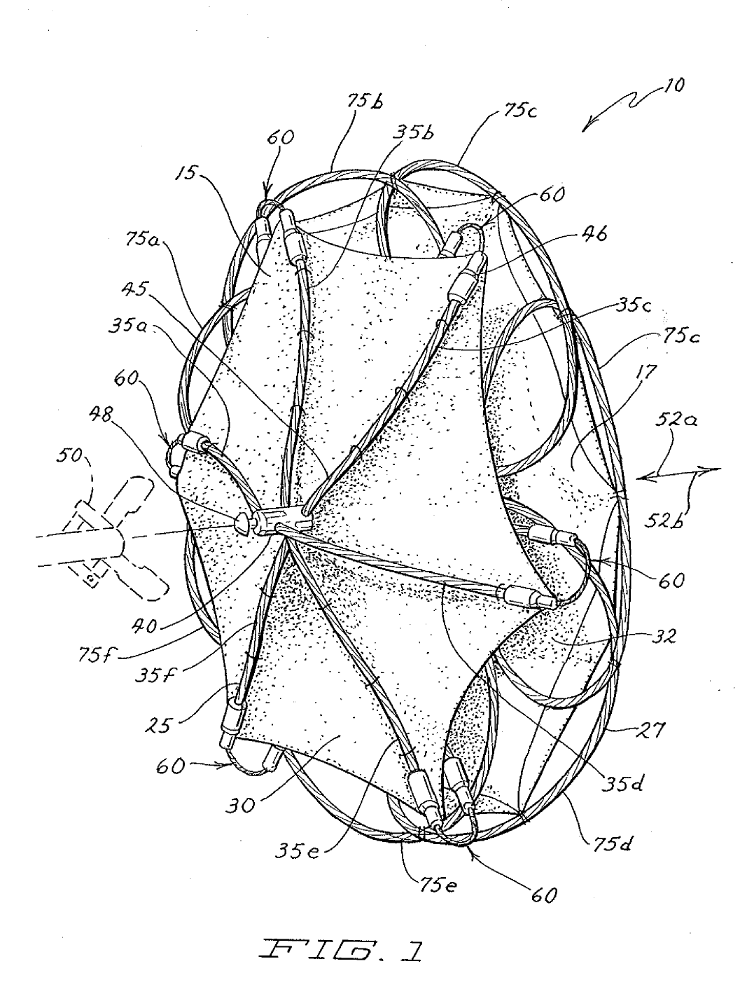

An exemplary embodiment of an occlusion device 10 is illustrated in FIG. 1. In this perspective view, the right side 15 of the device 10 is shown in the foreground and the left side 17 in the background. Throughout, the terms “right” and “left” are used for convenient reference and are selected in accord with the orientation of the device as it would typically be situated in the heart and in accord with typical cardiac terminology for distinguishing the sides of the heart. These terms should not, however, be considered limiting. (It is noted that these terms are opposite to the orientation of the device on the page in FIG. 1, such that the right side 15 of the device is on the left side of the page.) The device 10 includes right and left frames 25 and 27 respectively. A right sheet 30 is coupled to the right frame 25 and a left sheet 32 is coupled to the left frame 27.

### The Right Frame

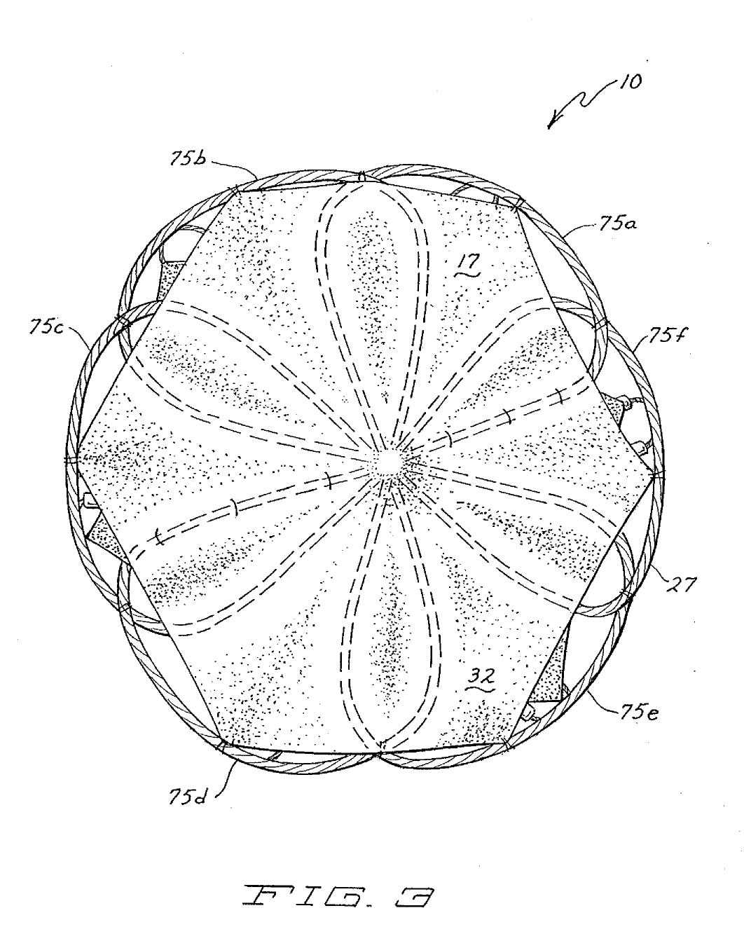

One embodiment of a right frame 25 is depicted in FIGS. 1-12. As depicted in FIG. 1, one embodiment of a right frame 25 is formed in part by several radially-extending arms 35a-35f. The right frame 25 is coupled to a deployment post 40; more specifically, one end of each arm, typified by central end 45 on arm 35c, connects to the deployment post 40. The arms 35a-f radiate from the deployment post 40 and terminate at their opposite ends, typified by terminating end 46 on arm 35c, adjacent the periphery of the device 10. The deployment post 40 terminates in a grasping knob 48 that can be grasped by a deployment tool 50 that is used to exert axial forces, in the directions indicated by arrows 52a-b, to selectively deploy and retract the device 10, as will be described below.

The right sheet 30 is connected to the arms 35a-f by, for example, folding a portion (such as a tab) of the sheet around the arm. This folded-over portion can then be laminated to the frame. Alternatively, the sheet 30 can be connected to the arms 35a-f by stitches at points along the length of some or all of the arms. In this exemplary embodiment, the sheet 30 is disposed on the interior side of the arms.

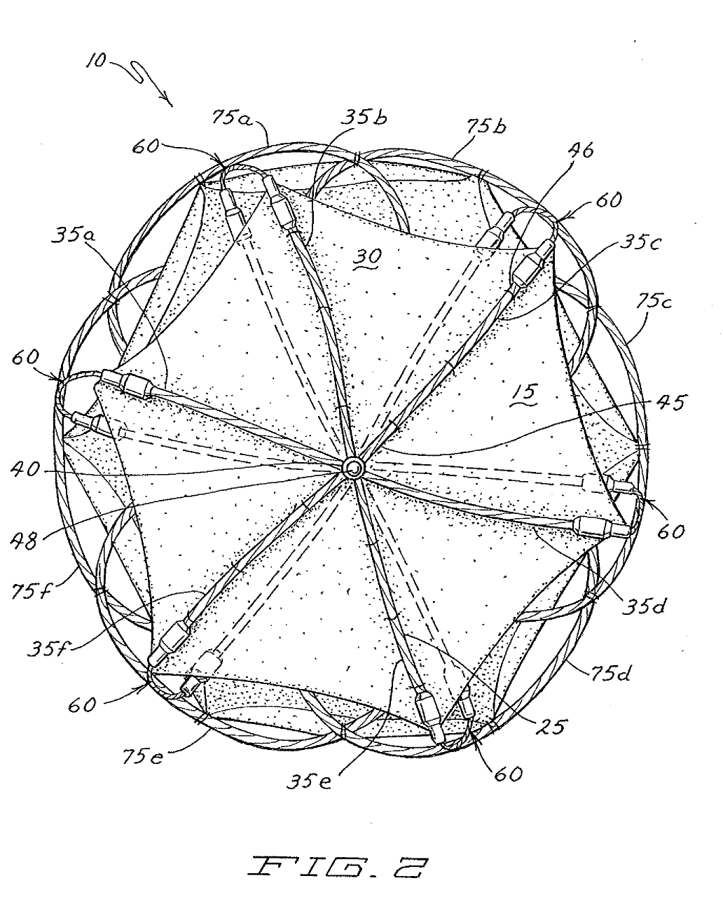

FIG. 2 shows the right frame 25 in an end view.

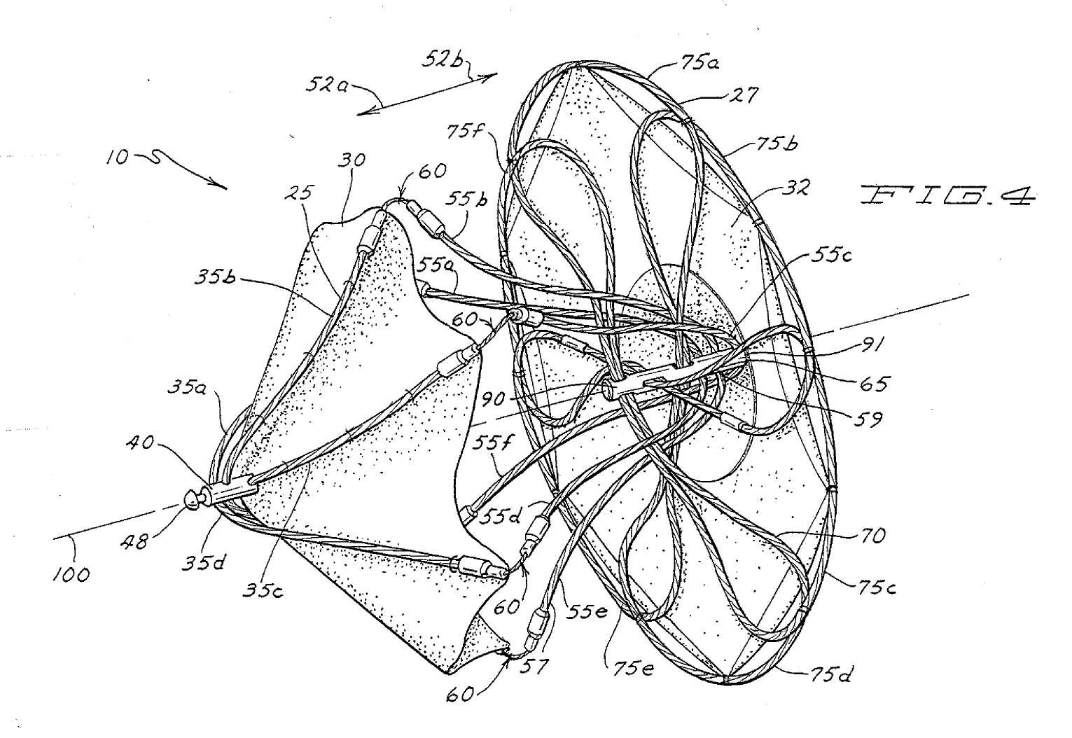

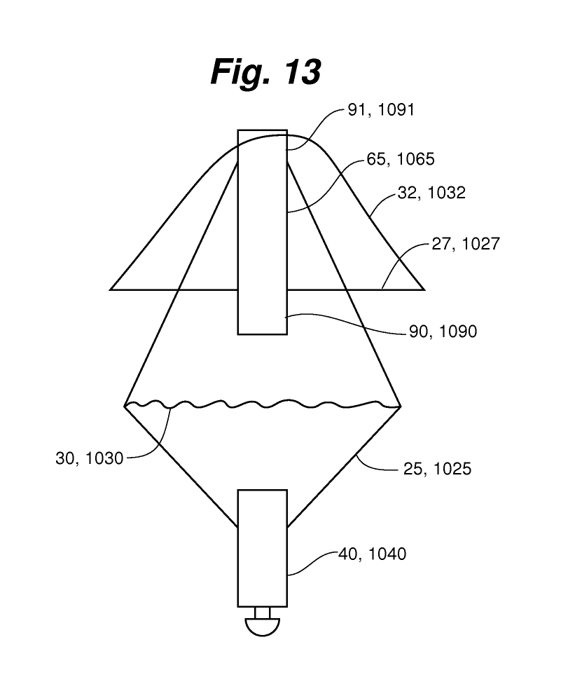

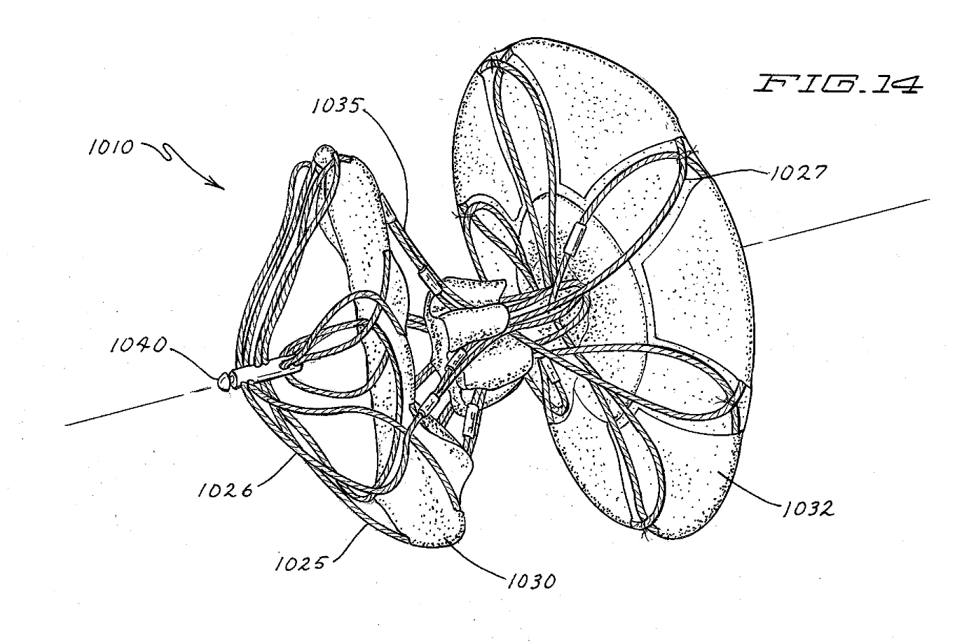

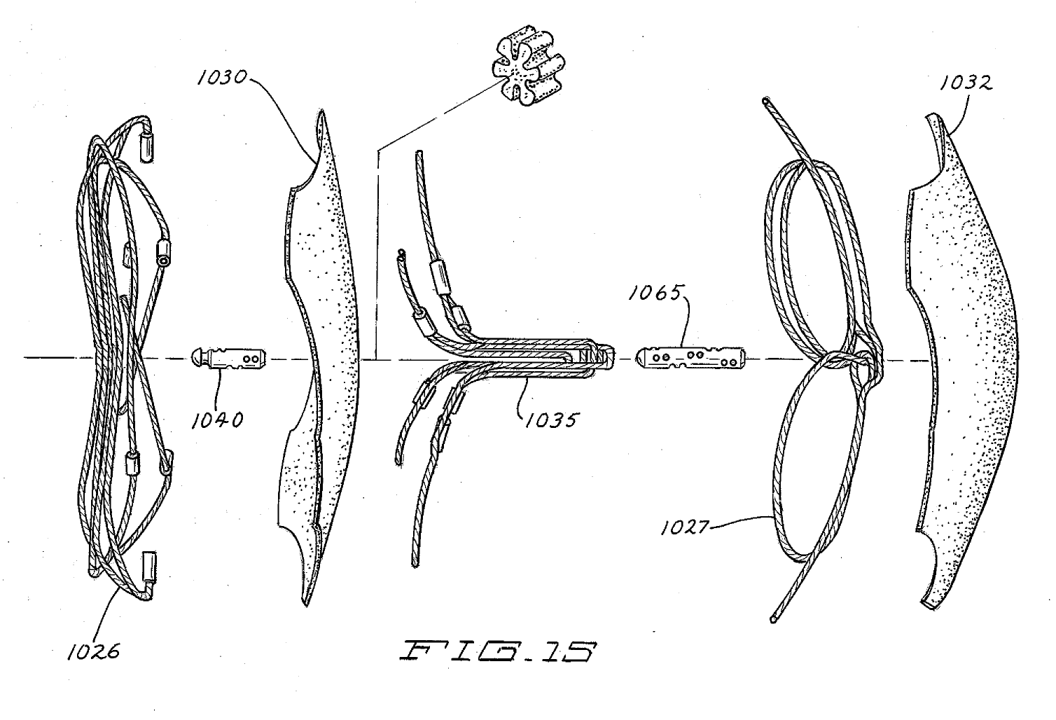

FIG. 4 reveals the structure of the device 10 between the sheets 30, 32. In addition to arms 35a-f, the right frame 25 includes elongate limbs 55a-f. These limbs 55a-f each have first and second opposite ends, typified by ends 57 and 59 on limb 55a. The limbs 55a-f are each coupled to a respective arm 35a-f via links, typified by link 60. These links 60 are couplings that allowing the limbs to fold with respect to the arms 35a-f. The links 60 will be described in greater detail below with respect to FIGS. 13a and 13b.

The opposite terminating ends 59 of the limbs 55a-f are coupled to a floating connecting post 65 in a manner that will be described in greater detail below.

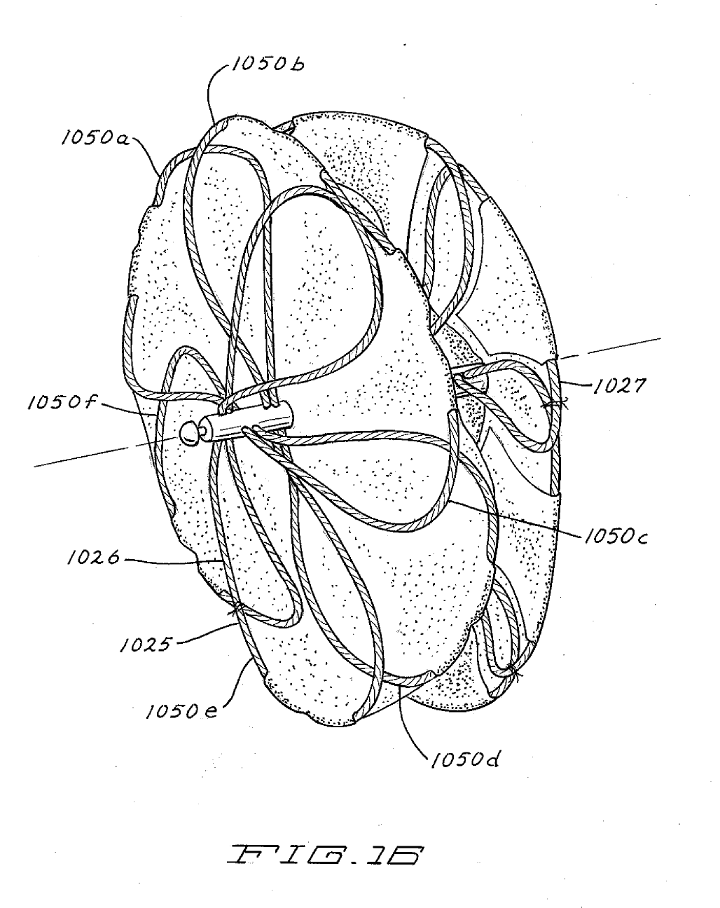

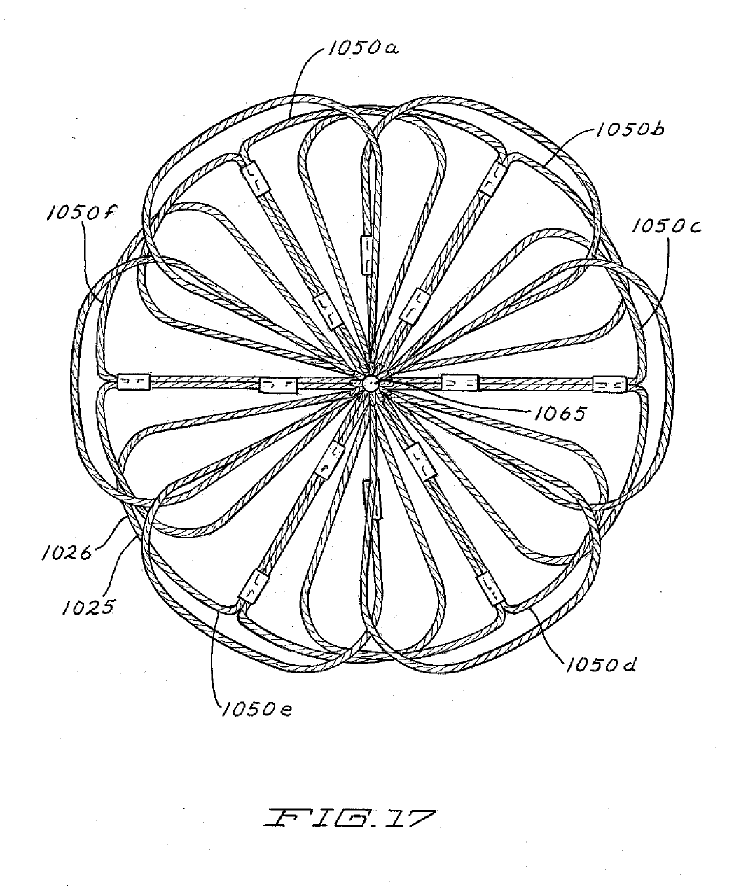

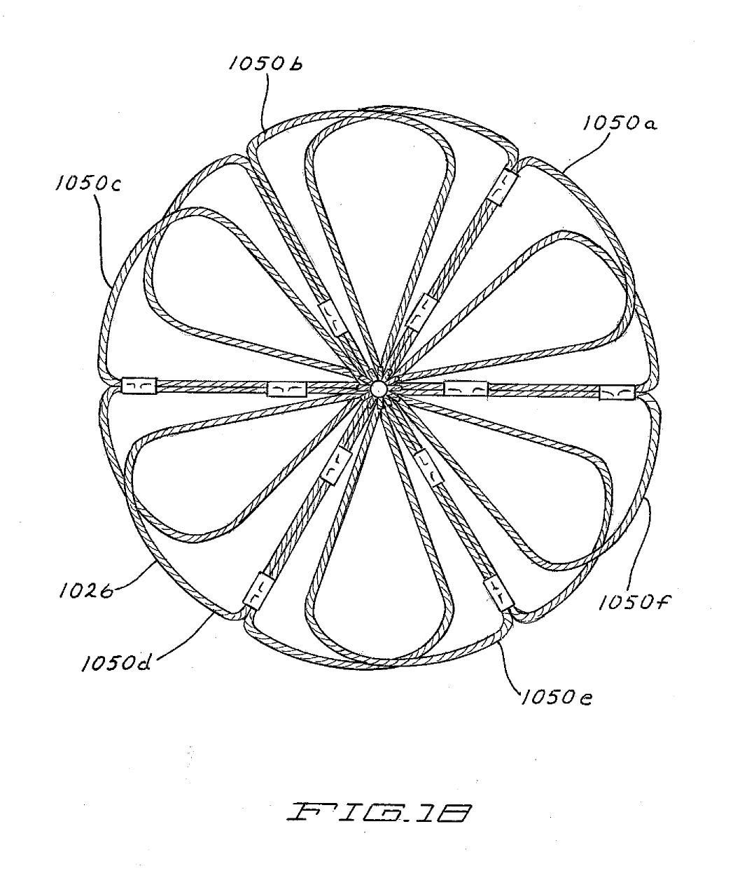

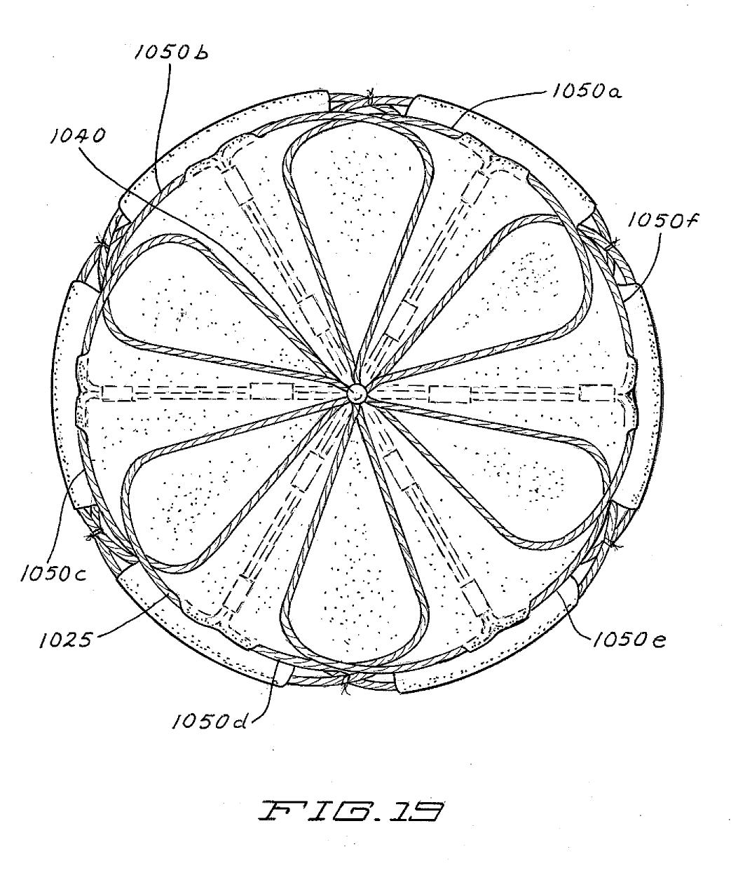

Another embodiment of a right frame 1025 is depicted in FIGS. 14-23, and will be described below.

### Left Frame

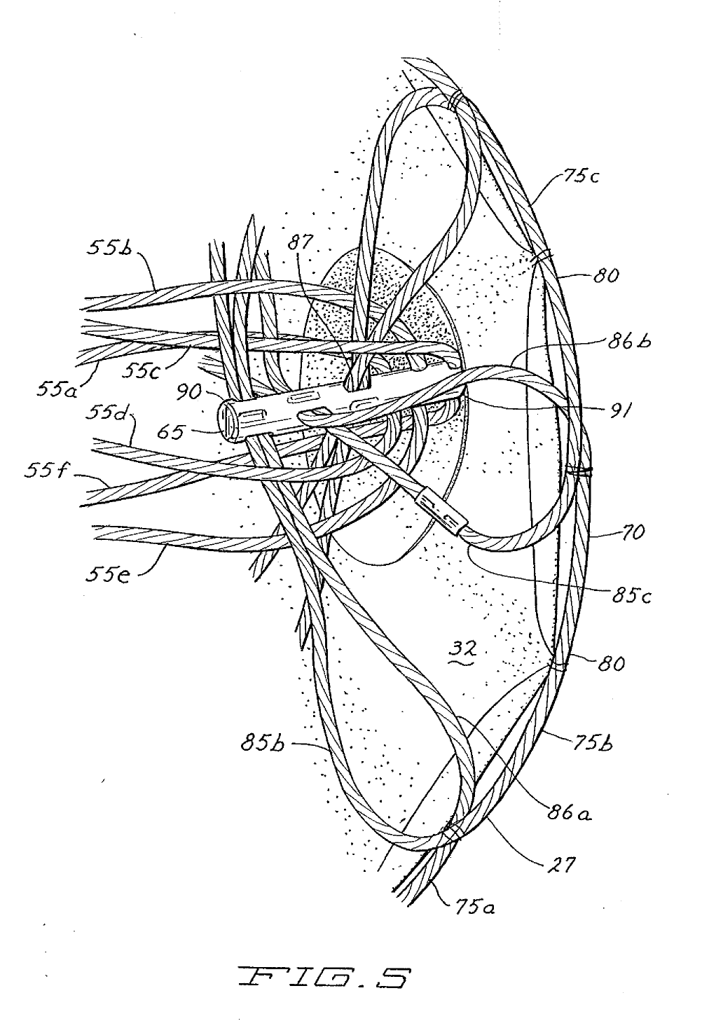

FIGS. 4 and 5 show the left frame 27 of the device 10. The left frame 27 is formed by a spline or splines 70 that form a series of overlapping loops or “petals” 75a-f that emanate or radiate from, and are coupled to, the connecting post 65. The radially outward-most portion 80 of each petal 75 defines the periphery of left frame 27. The left sheet 32 is connected to the left frame 27 by folding a portion (such as a tab) of the sheet around the frame and laminating the joint or by stitches at locations spaced about the periphery. In the exemplary embodiment illustrated, the sheet 32 is located on the exterior side of the frame 27. The petals 75a-f are interposed, such that one “edge” portion of a given petal overlaps and lies interior to the adjacent petal, while the opposite edge of the same petal overlaps and lies exterior to the opposite adjacent petal. This is apparent in FIG. 4 in which petal 75b lies between adjacent petals 75a and 75c. Left edge portion 85b of petal 75b overlaps and lies interior to right edge portion 86a of petal 75a. The right edge portion 86b of petal 75b overlaps and lies exterior to left edge portion 85c of petal 75c. This alternating over-under arrangement of adjacent petals provides stability and strength in the left frame 27, while still allowing sufficient flexibility to collapse to fit within a catheter.

The petals are formed by splines of any suitable material having the required strength and flexibility. One such suitable material is nitinol wire.

The multiple petals 75a-f of the left frame 27 can be formed of a single spline or multiple splines. In the exemplary embodiment depicted, the splines pass through apertures, typified by aperture 87, in the connecting post 65 and can be mechanically crimped to secure them. Several apertures 87 are axially spaced along the connecting post 65. Each petal is formed by a spline that exits the connecting post 65 at one location along the post's length and reenters at another location along the post's length, such that each petal is slightly askew or tilted. This aids in providing stability for the alternating over-under arrangement of adjacent petals.

The petal shapes of the splines 70 distribute forces relatively evenly about the periphery of the frame 27. This is advantageous because, in situ, the left frame 27 will not impart excessive force that would cause localized pinching or squeezing of adjacent tissue. Such pinching or squeezing at points in the tissue could prevent blood flow to the tissue and may damage the tissue. In addition, the uniform distribution of force about the periphery provides for effective and reliable occlusion, i.e. there are no locations of particularly weak force that would yield leak points. Still further, the petal shapes of the splines provide gentle curves to the periphery of the left frame 27 and that is advantageously atraumatic to tissue.

### The Connecting Post and Interleaved/Laced Frames

As shown in FIG. 4, the connecting post 65 has right and left opposite ends 90, 91, respectively. The limbs 55a-f connect to or pass through the connecting post 65 adjacent the post's left end 91; the splines 70 connect to or pass through the connecting post 65 adjacent the post's right end 90. In other words, the limbs 55a-f each connect to the connecting post 65 at positions on the post 65 that are further to the left than the positions on the post 65 to which the splines 70 connect. The result of these connecting positions is that the limbs 55a-f are laced with or are interleaved with or pass by the splines 70. One way of conceptualizing this arrangement is to imagine a plane through the post 65, perpendicular to the post's axis, between its left and right ends; both the splines 70 and the limbs 55a-f would pass through or intersect this plane. This aids in allowing the device to conform to a variety of defective geometries as will be described further below. Further, it aids in making the device easily collapsible for loading and reloading into a catheter.

Resiliency, Shape, and Range of Configurations (Natural, Deployed, in-Catheter)

Limbs 55a-f are formed of a resiliently deformable material, such as nitinol, in the form of wires or cables. In an exemplary embodiment, limbs 55a-f are subjected to pre-shaping to give them “shape memory” so that during manufacture, they are biased into a predetermined shape, even after undergoing deformation, such as when the device 10 is loaded in a catheter. One suitable shape for limbs 55a-f is a bell shape. This shape aids in allowing occlusion device 10 to maintain a low profile once the device 10 is deployed, and also allows limbs 55a-f to center the device 10 within a defect.

The device 10 is biased into its natural shape and configuration shown in FIGS. 1-3, in which the radial arms 35a-f of the left frame 27 extend radially outward, as do the petals 75a-f of the right frame 25, such that the arms 35a-f and the petals 75a-f form flanges 120, 121 that, in use, will sandwich tissue therebetween under slight force, as depicted in the schematic view presented in FIG. 6. Under slight axial force, the device 10 elongates slightly to accommodate tissue between the flanges 120, 121; under greater axial force, the device deforms to a collapsed configuration small enough to pass through a catheter for deployment, as will be described below. In addition, the flanges 120, 121 are constructed to provide flexibility to accommodate various defect sizes and geometries.

With further reference to FIG. 6, the device 10 positioned within a defect 92. In this in situ configuration, flanges 120, 121 are positioned on opposite sides of the defect with limbs 55a-f extending between the flanges 120, 121. More specifically, the connecting post 65 floats within the defect, and the limbs 55a-f connect thereto, as to the splines 70 of the left frame. The limbs 55a-f provide a flexible intermediate zone 93. Because the limbs 55a-f are flexible, the diameter of the intermediate zone 93 adjusts to the size and shape of the defect 92. The limbs 55a-f are biased to push outwardly to the largest diameter or periphery that the defect 92 will allow, thereby assuring that the device 10 is centered within the defect 92. (In FIG. 6, the limbs 55a and 55d are shown spaced from the tissue 94 that defines the defect 92; however, this is simply a limitation of a schematic drawing; in practice some or all of the limbs 55a-f would abut the tissue 94 adjacent the defect 92.) The biasing radially-outward force, in the direction indicated by arrow 95, supplied by the limbs 55a-f is strong enough to aid in centering the device 10 within the defect, but not strong enough to significantly displace tissue around the defect. Being properly centered increases the quality of the occlusion and thereby reduces the amount of blood that may shunt around the device 10, improving its therapeutic effect while the device 10 becomes endothelialized. Being properly centered also improves the odds of complete endothelialization.

In addition, the device 10 is resiliently deformable to allow it to increase and decrease in axial length, in the direction indicated by arrow 98 in its deployed configuration. In other words, the distance between the flanges 120, 121 or the sheets 30, 32 is varied to comply with thickness of the septum adjacent the defect. This axial length accommodation results at least in part from the flexibility in the limbs 55a-f. The limbs 55a-f move between a position in which they are roughly adjacent the center axis 100, such that the length 105 between the two sheets 30, 32 is maximized, to a position in which they splay radially outward such that the distance between the two flanges or sheets is minimized, as in FIG. 1. The limbs 55a-f are biased into the latter configuration where the distance is minimized. This bias aids the device 10 in sandwiching the tissue 110 that is adjacent the defect 89 between the flanges 120, 121 formed by the left and right frames, i.e. by exerting a force that pulls the flanges 120, 121 toward one another, thereby holding the device 10 in place until endothelialization takes place. A biased shape of the links 60, which may be resiliently deformable, may also contribute to biasing the device to its shortest axial length.

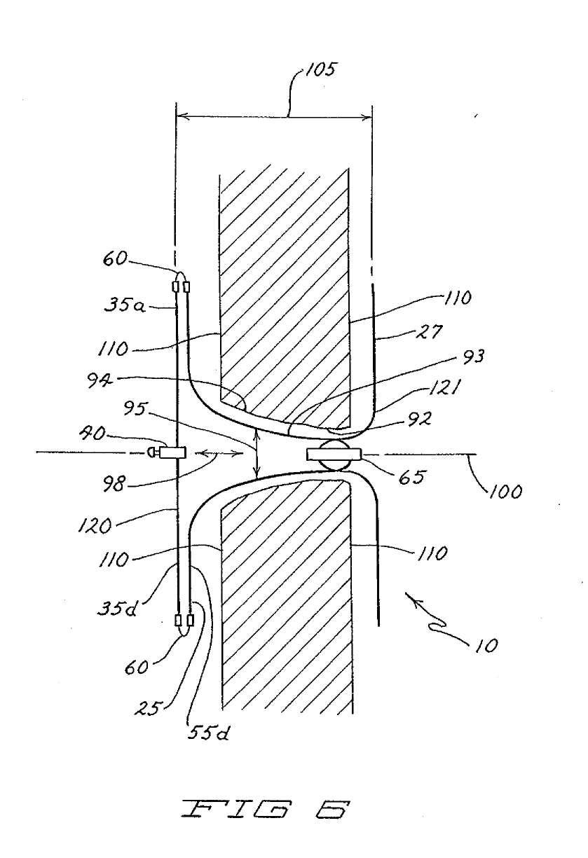

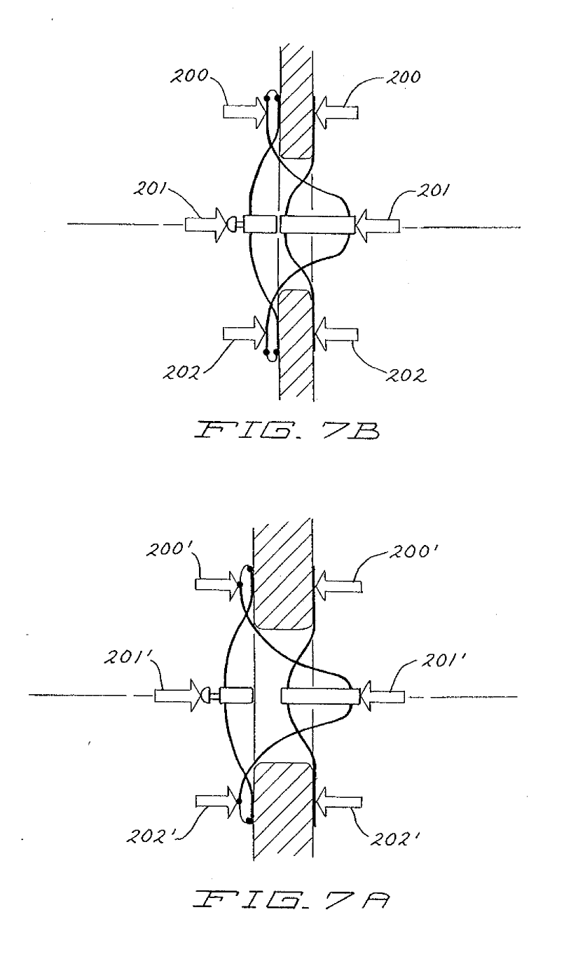

The schematics of FIGS. 7a and 7b depict the manner in which this design accommodates various wall thicknesses, as well as showing the benefits that result from the described device on force distribution on tissue adjacent the defect. The defective septum in FIG. 7a is thicker than the septum in FIG. 7b. To accommodate a thicker septum, the device 10 in FIG. 7a is expanded somewhat in its axial length. The sandwiching forces applied by the device 10 to the tissue adjacent the defect are depicted by arrows 200 and 201. More specifically, force is applied even at the radially-outermost portion of the device, aiding in holding the device 10 securely in place. Further, these forces are relatively uniform across the diameter of the device. That is, forces 200 are generally similar to forces 201 and 202. This results, in part, from the centering of the device within the defect; it results, further, from the device's natural bias, from the gentle curves of the limbs biased in a bell shape, from the interleaved configuration of the left and right frames, and from the disconnect between the post 40 to which the arms 35 are connected and the post 65 to which the splines 70 are connected allowing axial movement therebetween.

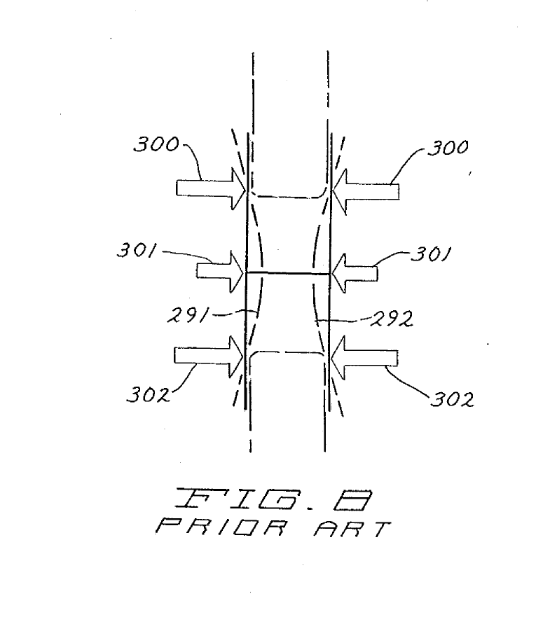

FIG. 8, in contrast, shows a prior art device that has a fixed length center post 290 extending between flanges 291, 292. The forces generated by this device are greatest at the corners of the tissue adjacent the defect. This concentration of forces 300, 302 at a particular spot in the tissue can prevent blood flow to the tissue and cause the tissue to degrade or die, thereby inhibiting occlusion. Further, the fixed post geometry offered no forces on the radial edges of flanges 291, 292, where it is most beneficial in securing the device 10.

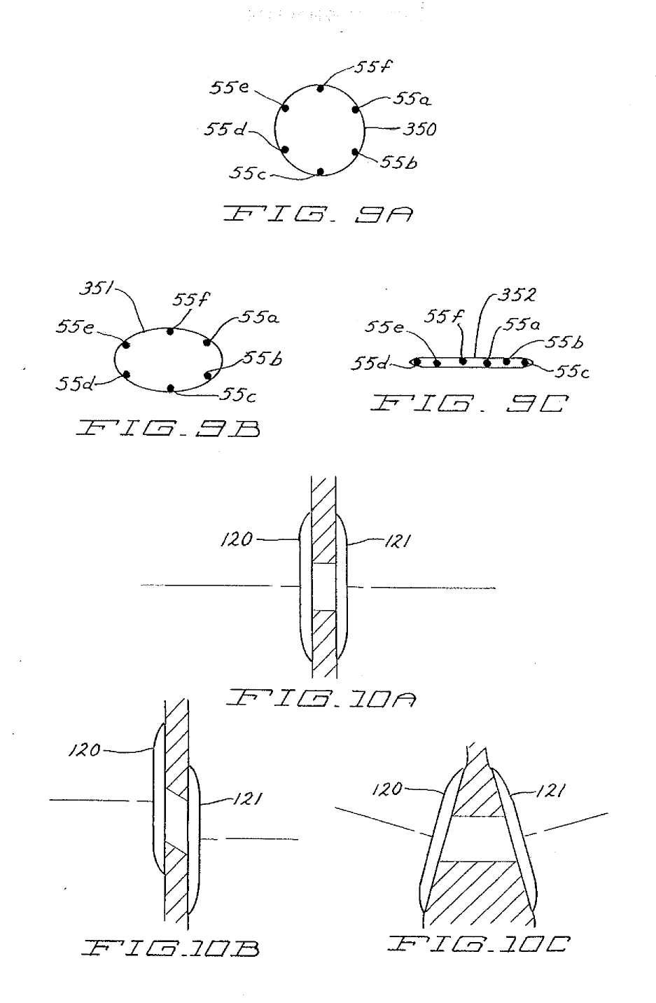

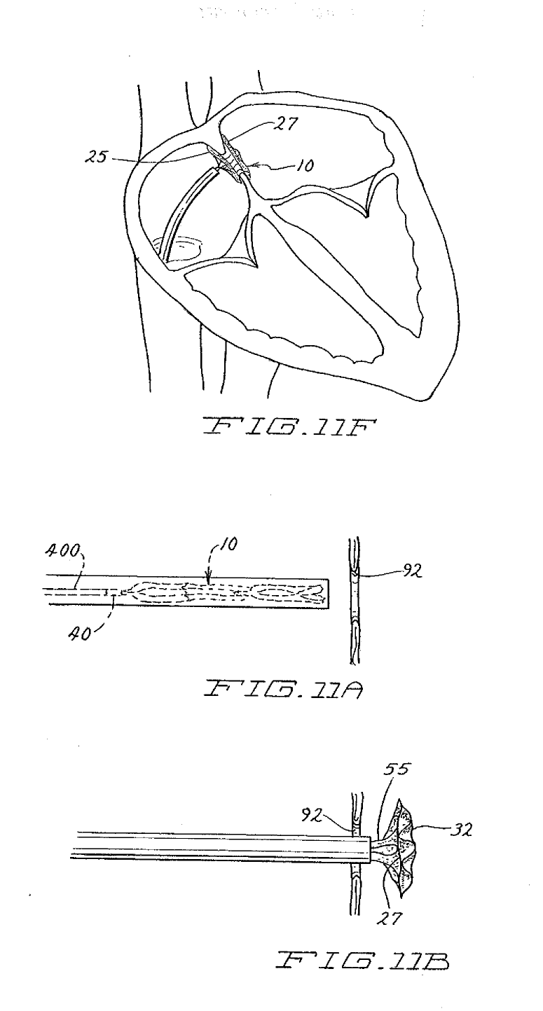

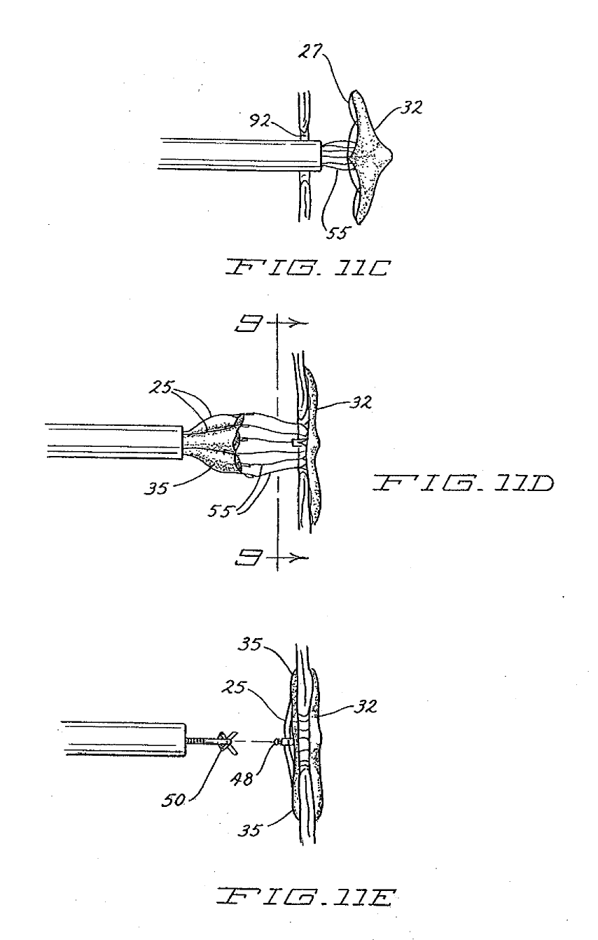

The sets of schematic drawings in FIGS. 9 and 10 show some of the flexibility and adaptability that result from the configuration of the present device 10 of FIGS. 1-4. More specifically, FIGS. 9a-9c depict a projection of the limbs 55a-f as they pass through defects of various shapes. FIG. 9a depicts a relatively circular defect 350; FIG. 9b depicts an oval-shaped defect 351; FIG. 9c depicts a defect that is very narrow or slit-shaped. The limbs 55a-f are able to conform to any of these shapes, from spreading to fill the circular shape of 350 to aligning in a single layer to fit with the slit 352.

FIGS. 10a-c further illustrate schematically how the device 10 accommodates various defect geometries. In FIG. 10b, the defect is skewed or slanted with respect to the adjacent wall; in this case, the device 10 allows for the flanges 120, 121 to similarly skew. In other words, the flanges 120, 121 have the freedom of movement to allow them to offset in their axial alignment and still provide a centered fit. FIG. 10c shows how the device 10 is able to adapt to another geometry in which the heart wall varies in thickness around the defect.

Of course, in real patients, the defects typically are defined by combinations of these alternative geometries to varying degrees and this device 10 is able to accommodate a wide range of these combinations, providing reliable occlusion where prior art devices previously did poorly or failed altogether. Further, by accommodating defects of various geometries and sizes, the device 10 yields efficiencies in manufacturing, inventory control and the like. Further, it decreases the number of devices used per procedure since the doctor need not use trial and error of a number of devices tailored to specific sizes and shapes of defects, spoiling rejected devices in the process; therefore, the cost per procedure is significantly reduced. Nevertheless, it is possible to tailor the device more particularly to various defect shapes and sizes by heat-shaping the limbs 55a-f accordingly.

### Deployment

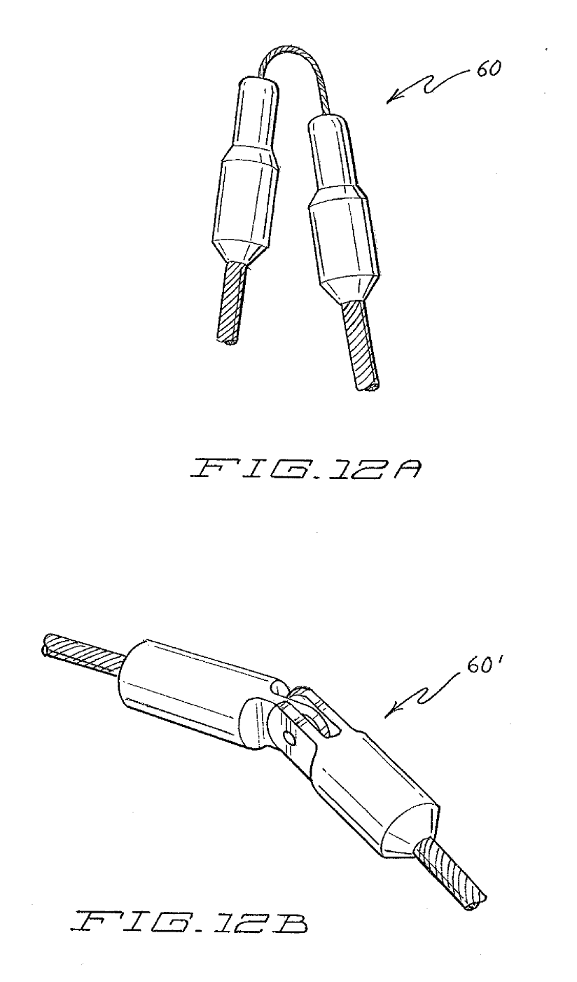

As noted, the device 10 can, under axial force, deform to a collapsed configuration to fit within a catheter for delivering the device to the defect site. FIGS. 11a-f depict in series how the device 10 is deployed. As shown in FIG. 11a, the device 10 in its collapsed state within a catheter and connected to a deployment wire 400 connected to a deployment post 40, is maneuvered into position adjacent the defect to be occluded. As depicted in FIG. 11b, the terminating end of the catheter is positioned on the opposite side of the defect 92. The device 10 is pushed partway out of the catheter, so that the left frame exits the catheter. The left frame, freed from the catheter, expands to its naturally-biased shape as shown in FIG. 11c. The operator snugs the left frame against the heart wall adjacent the defect and then continues to expel the device from the catheter, FIG. 11d. When the right frame is freed from the catheter, it adopts its naturally-biased configuration, shown in FIG. 11e. The operator disconnects the deployment wire 400 from the device 10, as shown in FIG. 11f.

Although an illustrative version of the device is shown, it should be clear that many modifications to the device may be made without departing from the scope of the invention. For example, two exemplary embodiments of the links 60, 60′ are depicted in FIGS. 12a and 12b. In an exemplary embodiment of FIG. 12a, a link 60 are made of a relatively small-diameter wire to provide for a relatively sharp, or small radius-of-curvature, bend. In the exemplary embodiment of FIG. 12b, the link 60′ is a hinge about an axis. In an alternative embodiment of a link not shown, associated limbs and arms might each be formed of a unitary member with a transition region between the limb portion and the arm portion that may have different strength or flexibility properties than the limb and arm portions. By joining the arms and limbs via links or transition regions, optimal choices can be made to provide the desired strength in the limbs and arms while achieving flexibility in the joints or transition therebetween.

### Schematic

FIG. 13 is a skeletal schematic that depicts, conceptually, the organization of components in this device 10, that has been described above in greater detail. A connecting post 65 has right and left ends 90, 91, respectively. A left frame 27 is coupled to a connecting post 65, adjacent the right end 90 of the post 65. A deployment post 40 is separate from and roughly longitudinally-co-axial with or longitudinally-aligned with the connecting post 65. (The alignment of the posts 40, 65 in situ will be determined by the geometry of the defect.) A right frame 25 is coupled to the deployment post and to the connecting post 65 adjacent its left end 91. The right frame supports a right sheet 30; the left frame 27 supports a left sheet 32.

Another Embodiment with Different Arrangement for Right Frame

FIGS. 14-23 show a version of the device 1010 that shares many similarities with device 10, but has an alternative arrangement for the right frame 1025. Device 1010 has the same arrangement conceptually as device 10 as is depicted in FIG. 13. That is, device 1010 has a connecting post 1065 with right and left ends, 1090, 1091, respectively. A left frame 1027 is coupled to a connecting post 1065, adjacent the right end 1090 of the post 1065. A deployment post 1040 is separate from and roughly longitudinally-co-axial with or longitudinally-aligned with the connecting post 1065. (The alignment of the posts 1040, 1065 in situ will be determined by the geometry of the defect, but they are biased, at rest, roughly into longitudinal alignment.) A right frame 1025 is coupled to the deployment post and to the connecting post 1065 adjacent its left end 1091. The right frame supports a right sheet 1030; the left frame supports a left sheet 1032.

The right frame 1025 includes a sheet-support portion 1026, for supporting the right sheet 1030 and a limb portion 1035 that couples the right frame 1025 to the connection post 1065 and that in situ, passes through the defect. The sheet-support portion 1026 defines a generally circular circumferential edge to which the radially-outer edge of the sheet 1030 attaches at intervals, providing radially-outward forces on the sheet to keep it spread across the defect in situ. The sheet-support portion 1026 is formed of an array of petals 1050a, b, c, d, e, and f. These petals appear in FIGS. 14, 15, 16, 17, 18, 19. The petals overlap one another.

The limb portion 1035 is to the left of the right sheet 1030; that is, the limb portion 1035 extends between the two sheets 1030, 1032. As with device 10, the limb portion 1035 of device 1010 interleaves with the left frame 1027 because the limb portion 1035 is coupled to connecting post 1065 adjacent its left end 1091, while the left frame 1027 is coupled to the connecting post 1065 adjacent the connecting post's right end 1090.

In the illustrated embodiment, wires of the right frame 1025 form six petals 1050a-f and six limbs 1052a-f. Because of the manner of looping and coupling of these wires, described in greater detail below, each petal 1050 is formed by two subpetals that are formed from portions of two looped, coupled wires. Similarly, each limb has two separate wires coupled together.

The petals 1050a-f provides a gently curved outer, circumferential edge that roughly defines a circle. This is advantageous because it has no pointed corners or joints that might cause injury. The right sheet 1030 attaches at its circumferential edge to the circumferential edges of the petals. The radially outward force on the right sheet can be relatively uniform since the petals provide a nearly circumferential edge pulling outward on the sheet. Still further, the elegant design minimizes the dangers of tangling of the wires during deployment and redeployment.

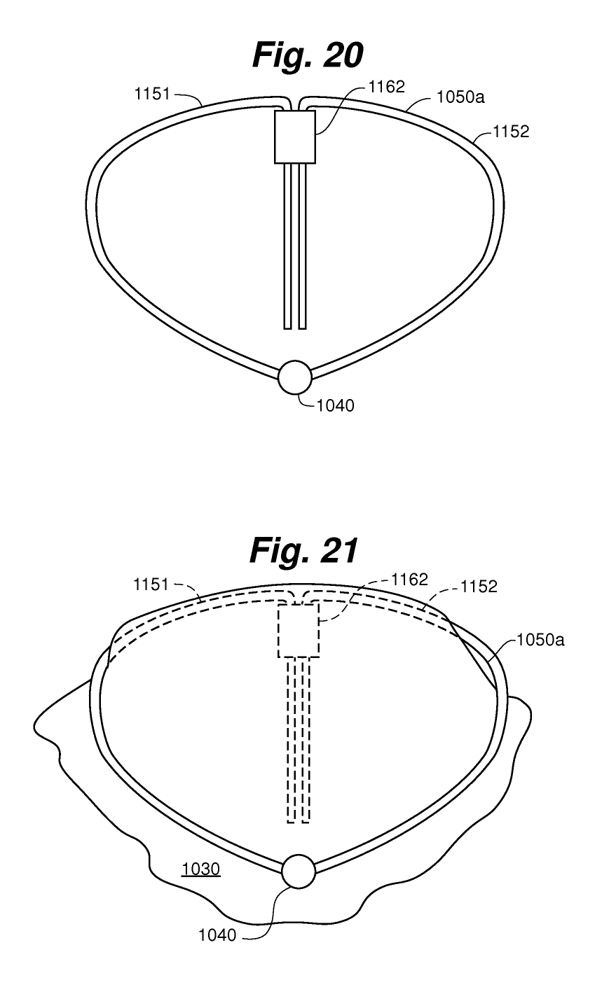

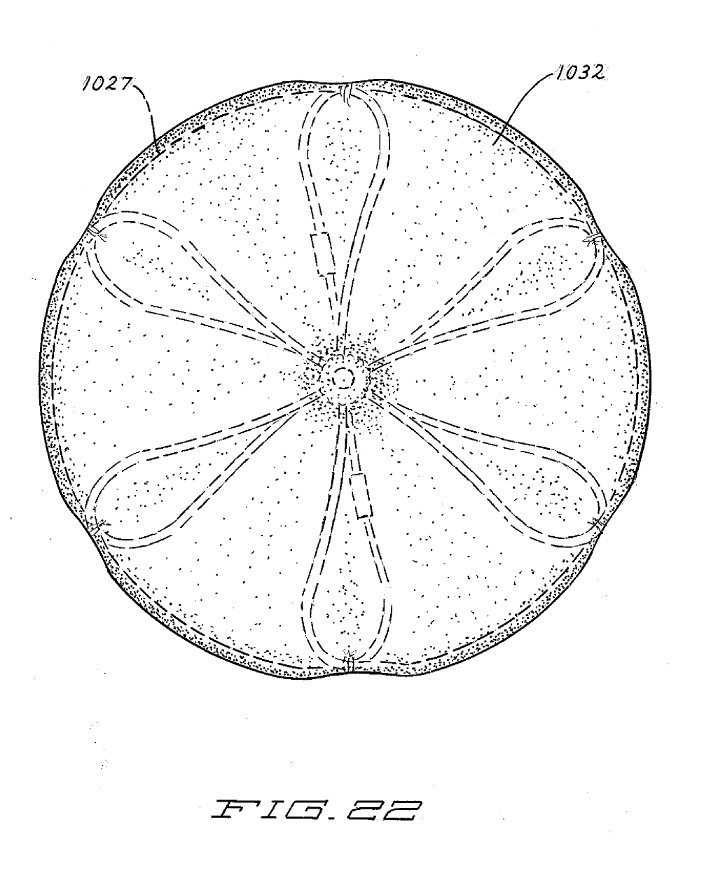

FIGS. 20 and 21 each show one petal of the right frame 1025, in an enlarged, partial view. In FIG. 20, shows petal 1050 without the sheet 1030; FIG. 21 shows the petal 1050 with the sheet 1030 attached. The petal 1050 is formed from portions of two wires 1151, 1152, that are joined together by a collar 1162.

The device illustrated in FIGS. 14-25 is configured for use to occlude a patent foramen ovale (PFO) defect. The geometry of a PFO is best occluded with a device that is relatively narrow, i.e. of a small diameter or with all limbs relatively close together, through the limb portion 1035. The narrowness of the limb portion results from the form into which it is biased during manufacture, as will be described below. Devices for occluding other types of defects may be optimized with slightly different geometries. For example, a device for occluding an atrial septal defect will have slightly wider limb portion.

### Construction of the Alternate Arrangement of Right Frame

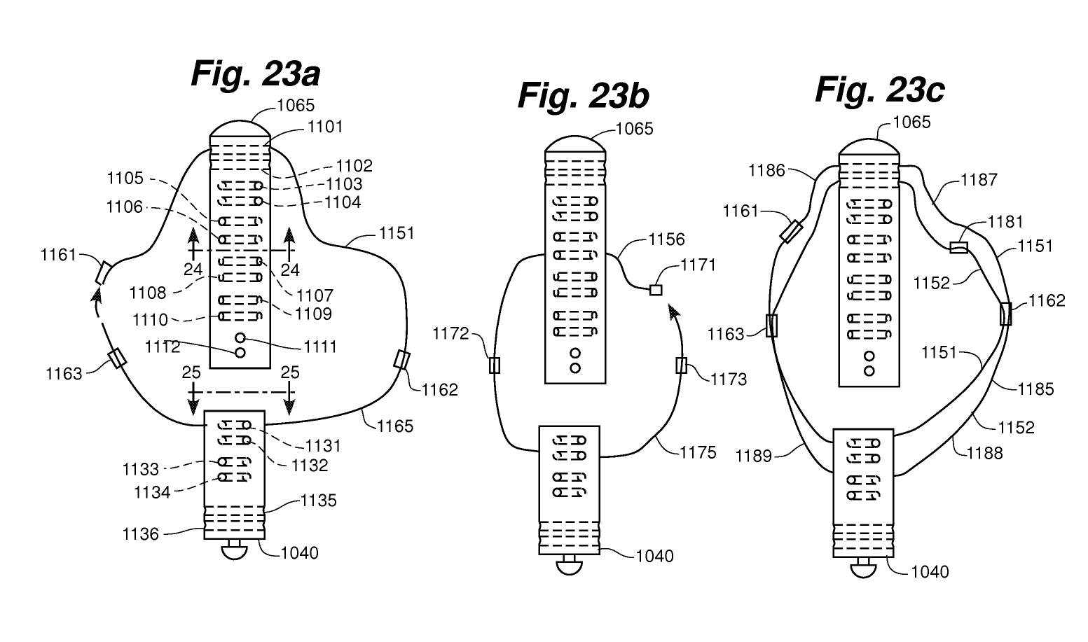

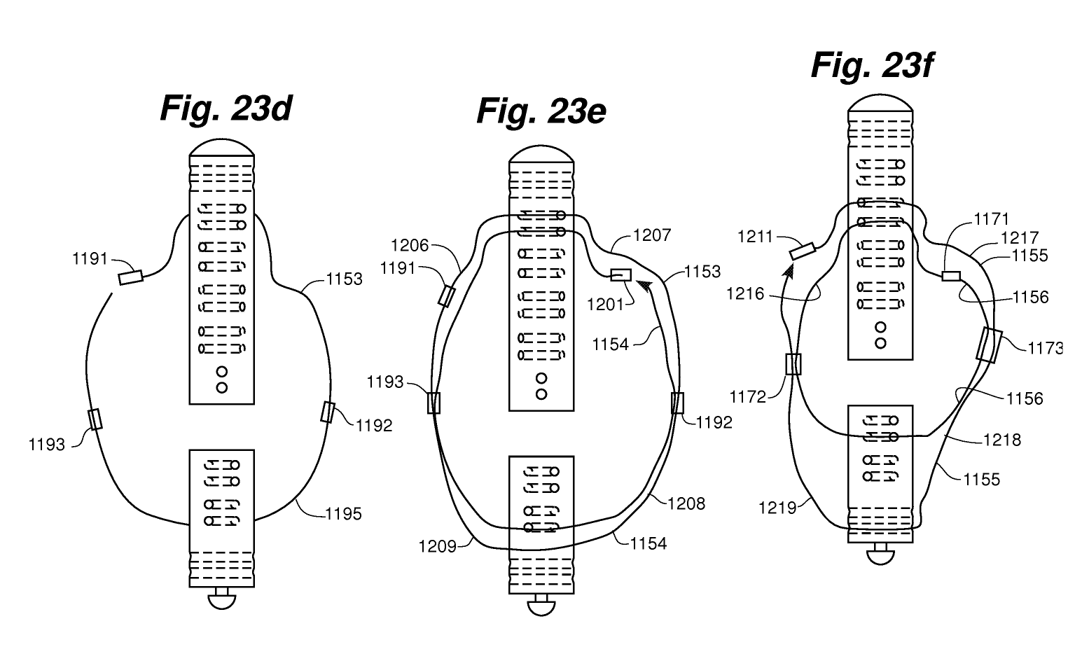

In this embodiment, a series of looped wires form both the sheet-support portion and the contiguous limb portion 1035. The loops are formed by passing the wires through predefined apertures in the connection post and the deployment post. This is depicted in FIG. 23a-f. Six wires are cut to desired lengths. Then, for each wire, the following steps are followed:

a) Near one end (i.e. by definition the “short” end) of each wire, a centering curve is formed;

b) an end cap or coupler is placed on the short end of the wire;

c) the long end (by definition, the end opposite the short end) of the wire is passed through an aperture in the connecting post;

d) a collar is slipped over the long end of the wire (or the wire is passed

through the collar of another wire, as will be described below);

e) the long end is passed through an aperture in the deployment post;

f) a second collar is slipped onto or over the long end of the wire (or the wire is passed through the collar of another wire as will be described below); and

g) the long end is inserted in the end cap or coupler on the short end to join the two ends.

Thus, once installed, each wire follows a path through the connecting post, through a collar, through the deployment post, through a second collar and then its ends are joined, forming a loop.

The wires are installed in a predetermined order and through predetermined post apertures. This is depicted in FIGS. 23a-f. (These simplified figures are presented to illustrate the order of wire installation; therefore, for simplicity and clarity, only one or two wires are depicted in each figure, although other previously-installed wires are present. For example, before the wires that form the right frame are installed, the wires forming the left frame are installed on the connecting post; for simplicity the left frame wires are not shown.)

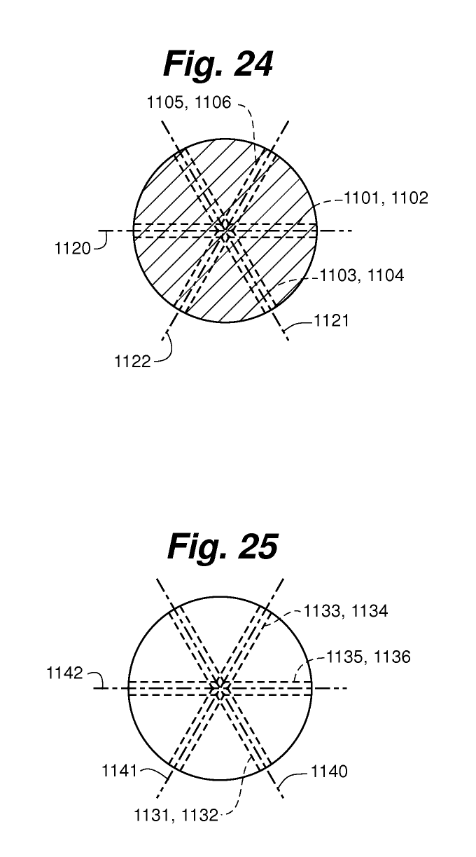

As shown in FIG. 23a, the connecting post defines twelve apertures, passing through the post. These apertures are numbered 1101 (for the left-most aperture, at the top of the post as it is oriented in the FIG. 23 series) through 1112 (for the right-most aperture, at the bottom of the post as it is oriented in the FIG. 23 series). The apertures 1101-12 are spaced from one another along the length of the post and enter/exit the post from various angles. As shown in FIG. 24, an enlarged cross section, taken along line 24-24 in FIG. 23a, apertures 1101 and 1102 are parallel to each other and follow a first trajectory 1120 through the post; apertures 1103 and 1104 are parallel to each other and follow a second trajectory 1121 through the post that is 60 degrees from trajectory 1110; and apertures 1105 and 1106 are parallel to each other and follow a third trajectory 1122 through the post that is 60 degrees from trajectory 1110 and 120 degrees from trajectory 1111. Apertures 1101-1106 accommodate wires that form the right frame, and apertures 1107-1112 accommodate wires that form the left frame.

Similarly, the deployment post 1040 defines a series of apertures 1131-1136, spaced from one another and arrayed along the length of the post. They enter/exit the post from various angles. As shown in FIG. 25, an enlarged end view, taken along line 25-25 in FIG. 23a, apertures 1131 and 1132 are parallel to each other and follow a first trajectory 1140 through the post; apertures 1133 and 1134 are parallel to each other and follow a second trajectory 1141 through the post that is 60 degrees from trajectory 1140; and apertures 1135 and 1136 are parallel to each other and follow a third trajectory 1142 through the post that is 60 degrees from trajectory 1140 and 120 degrees from trajectory 1141. Apertures 1131-1136 accommodate wires that form the right frame.

To form the right frame 1025 depicted in FIGS. 14-22, six wires 1151-1156 are installed. As shown in FIG. 23a, a coupler 1161 is placed on the short end of a first wire 1151; the long end of the first wire 1151 is passed through aperture 1101 in the connection post. Thereafter, a collar 1162 is slipped onto the long end of wire 1151. Next, the wire 1151 is passed through aperture 1131 in the deployment post. A second collar 1163 is slipped on to the long end of the wire 1151. Finally, the long end of the wire 1151 is joined to the short end via the cap 1161. In this manner, wire 1151 has formed a loop 1165 that passes through the connection post 1065 and the deployment post 1040.

As shown in FIG. 23b, a cap 1171 is placed on the short end of the next wire 1156 to be installed; the long end of the wire 1156 passed through aperture 1106 in the connection post. Thereafter, a collar 1172 is slipped onto the long end of wire 1156. Next, the wire 1156 is passed through aperture 1132 in the deployment post. A second collar 1173 is slipped on to the long end of the wire 1156. Finally, the long end of the wire 1156 is joined to the short end via the cap 1171. In this manner, wire 1156 has formed a loop 1175 that passes through the connection post 1065 and the deployment post 1140.

As shown in FIG. 23c, a cap 1181 is placed on the short end of the next wire 1152 to be installed; the long end of the wire 1152 is passed through aperture 1102 in the connection post. Thereafter, wire 1152 is slipped through first collar 1162 on wire 1151, thereby coupling wire 1152 to wire 1151. Next, the wire 1152 is passed through aperture 1133 in the deployment post. The wire 1152 is slipped through second collar 1163 on the first wire 1151. Finally, the long end of the wire 1152 is joined to the short end via the cap 1181. In this manner, wire 1152 has formed a loop 1185 that passes through the connection post 1065 and the deployment post 1140 and is coupled to wire 1151 at the two collars 1162, 1163. Four subloops are formed in the manner. Subloop 1186 is defined by wires 1151 and 1152 between collar 1163 and connection post 1065; subloop 1187 is defined by wires 1151 and 1152 between connection post 1065 and collar 1162; subloop 1188 is defined by wires 1151 and 1152 between collar 1162 and deployment post 1040; subloop 1189 is defined by wires 1151 and 1152 between deployment post 1040 and collar 1163.

As shown in FIG. 23d, a cap 1191 is placed on the short end of the next wire 1153 to be installed; the long end of the wire 1153 is passed through aperture 1103 in the connection post. Thereafter, a collar 1192 is slipped onto the long end of wire 1153. Next, the wire 1153 is passed through aperture 1134 in the deployment post. A second collar 1193 is slipped on to the long end of the wire 1153. Finally, the long end of the wire 1153 is joined to the short end via the cap 1191. In this manner, wire 1153 has formed a loop 1195 that passes through the connection post 1065 and the deployment post 1040.

As shown in FIG. 23e, a coupler 1201 is placed on the short end of the next wire 1154 to be installed; the long end of the wire 1154 is passed through aperture 1104 in the connection post. Thereafter, wire 1154 is slipped through collar 1192 on wire 1153, thereby coupling wires 1154 and 1154. Next, the wire 1154 is passed through aperture 1135 in the deployment post. Wire 1154 is slipped through collar 1193 on wire 1153. Finally, the long end of the wire 1154 is joined to the short end via the coupler 1201. In this manner, wire 1154 has formed a loop 1205 that passes through the connection post 1065 and the deployment post 1040 and is coupled to wire 1153 at the two collars 1192, 1193. Four subloops are formed in the manner. Subloop 1206 is defined by wires 1153 and 1154 between collar 1193 and connection post 1065; subloop 1207 is defined by wires 1153 and 1154 between connection post 1065 and collar 1192; subloop 1208 is defined by wires 1153 and 1154 between collar 1192 and deployment post 1040; subloop 1209 is defined by wires 1153 and 1154 between deployment post 1040 and collar 1193.

As shown in FIG. 23f, a coupler 1211 is placed on the short end of the next wire 1155 to be installed; the long end of the wire 1155 is passed through aperture 1105 in the connection post. Thereafter, the long end of wire 1155 is passed through collar 1173 on wire 1156, thereby coupling wires 1155 and 1156. Next, the wire 1155 is passed through aperture 1136 in the deployment post. Wire 1155 is passed through collar 1172 on wire 1156. Finally, the long end of the wire 1155 is joined to the short end via the coupler 1211. In this manner, wire 1155 has formed a loop 1215 that passes through the connection post 1065 and the deployment post 1140. Four subloops are formed in the manner. Subloop 1216 is defined by wires 1155 and 1156 between collar 1172 and connection post 1065; subloop 1217 is defined by wires 1155 and 1156 between connection post 1065 and collar 1173; subloop 1218 is defined by wires 1155 and 1156 between collar 1173 and deployment post 1040; subloop 1219 is defined by wires 1155 and 1156 between deployment post 1040 and collar 1172.

For convenient reference, the following table shows the reference numbers for the components of the right frame 1025.

| | | | Aperture | | Aperture | | | |
|---|---|---|---|---|---|---|---|---|
| | | | through | | through | | | |
| | | | Connection | First | Deployment | Second | | |
| Order | Wire | Coupler | Post | collar | Post | Collar | Loop | Subloops |
| 1 | 1151 | 1161 | 1101 | 1162 | 1131 | 1163 | 1165 | 1186-1189 |
| 2 | 1156 | 1171 | 1106 | 1172 | 1132 | 1173 | 1175 | 1216-1219 |
| 3 | 1152 | 1181 | 1102 | 1163 | 1133 | 1162 | 1185 | 1186-1189 |
| 4 | 1153 | 1191 | 1103 | 1192 | 1134 | 1193 | 1195 | 1206-1209 |
| 5 | 1154 | 1201 | 1104 | 1193 | 1135 | 1192 | 1205 | 1206-1209 |
| 6 | 1155 | 1211 | 1105 | 1173 | 1136 | 1172 | 1215 | 1216-1219 |

The wires used in the device are preferably a memory-shape wire, such as Nitinol, is used that can be positioned into the desired finished, biased configuration, then heated to a predetermined temperature for a given period of time, such that the wire frames take on the desired shape, or are biased into the desired shape, at a range of temperatures including room temperature.

After the wires are installed through the posts, the device is positioned in a jig, such that the loops are constrained within a circle of a specified radius, and such that the device is in the desired biased configuration. The collars 1162, 1163, 1172, 1173, 1192, 1193 are positioned as close to the perimeter as possible, creating a relatively sharp bend in the wires immediately adjacent the collars. (This juncture of the two wires is depicted, as discussed above, in FIGS. 20 and 21.) In the desired biased shape, the sheet-support portion is flange-like, and the limb portion tends toward the longitudinal center of the device.

While the invention has been described in connection with specific embodiments thereof, it will be understood that it is capable of further modification, and this application is intended to cover any variations, uses, or adaptations of the invention following, in general, the principles of the invention and including such departures from the present disclosure as come within known or customary practice in the art to which the invention pertains and as may be applied to the essential features hereinbefore set forth, and as fall within the scope of the invention and the limits of the appended claims. Further, terms such as “left” and “right” are used solely for convenient reference, but should not be deemed limited. Similarly, the labels of “deployment” and “connecting” to the two posts is descriptive for the embodiment depicted, but it should be appreciated that the function of these two posts could be swapped in some embodiments or uses and therefore should not be limiting.

I claim:

1. A device for occluding a defect in a heart wall, comprising:
a) a deployment post;
b) a connecting post having left and right ends;
c) a left frame coupled to said connecting post;
d) a right frame coupled to said connecting post and to said deployment post;
e) said right frame coupled to said connecting post adjacent the post's left end and said left frame coupled to said connecting post adjacent the connecting post's right end, such that said left and right frames are interleaved.

2. A device according to 1 wherein said right frame includes:
i) a sheet-support portion; and
ii) limb portion coupled to said sheet support portion and extending between said sheet support portion and said connecting post.

3. A device according to claim 2 wherein said sheet support portion is formed by splines arrayed in a series of petals.

4. A device according to claim 3 wherein each said petal of said right frame overlaps adjacent petals.

5. A device according to claim 3 further comprising:
f) a left sheet coupled to said left frame:
g) a right sheet coupled to said right frame.

6. A device according to claim 5, wherein an outer edge of said right sheet folds over the radially distal portion of said petals.

7. A device according to claim 1, wherein said right frame is resiliently deformable and is biased toward a first deployed configuration in which said connecting and deployment posts are in close proximity and further wherein said right frame is deformable under applied force to elongate thereby distancing said right frame from said left frame under tension to accommodate heart walls of various thickness and to squeeze heart wall tissue adjacent the defect slightly to hold said device in place.

8. A device according to claim 2, wherein said sheet-supporting portion is contiguous with said limb portion.

9. A device according to claim 1 wherein said right frame is formed by wires, each wire having first and second opposite ends coupled together such that the wire forms a loop and wherein said loop passes through apertures in said connecting post and said deployment post.

10. A device according to claim 5, wherein part of each said wire loop forms a sheet-supporting petal and wherein part of each said wire forms a limb portion.

11. A method of forming a device for occluding a defect in the heart, comprising the steps of:
a) providing first and second posts, said first post having left and right opposite ends;
b) forming a left frame coupled to said first post;
c) forming a right frame coupled to said first and second posts, wherein said right frame is coupled to said first post adjacent the post's left end and said left frame is coupled to said first post adjacent the first post's right end, such that said left and right frames are interleaved.

12. A method according to claim 11, wherein said left and right frames are formed of resiliently deformable wires.

13. A method according to claim 12, wherein said posts define apertures through which said wires pass.

14. A method according to claim 13, wherein the step of forming a right frame further comprises the steps of:
d) providing six wires;
e) for each wire:
i) forming a centering curve near said short end of the wire;
ii) placing an end cap or coupler on the short end of each wire;
iii) passing the long end of the wire through an aperture in the first post;
iv) slipping a collar over the long end of the wire;
v) passing the long end of the wire through an aperture in the second post;
vi) slipping a second collar onto or over the long end of the wire; and
vii) inserting the long end of the wire in the end cap or coupler on the short end to join the two ends.
